(19) United States
(12) Patent Application Publication
(10) Pub. No.: US 2022/0412935 A1
(43) Pub. Date: Dec. 29, 2022

(54) ANTIGEN TESTING

(71) Applicant: IgGenix, Inc., South San Francisco, CA (US)

(72) Inventors: Kari Nadeau, South San Francisco, CA (US); Derek Croote, South San Francisco, CA (US)

(21) Appl. No.: 17/847,385

(22) Filed: Jun. 23, 2022

Related U.S. Application Data
(60) Provisional application No. 63/214,298, filed on Jun. 24, 2021.

(51) Int. Cl. G01N 33/02; G01N 33/53; G01N 33/68

(52) U.S. Cl. CPC G01N 33/02; G01N 33/5308; G01N 33/6803

(57) ABSTRACT

The invention provides methods of testing food for antigens that are more stable to food processing than more clinically problematic allergens from the same food. When clinically-significant allergens are disproportionately broken down by common preparation methods, the presence of certain food ingredients may be “masked” to some tests yet may still be allergenic. To prevent false negative results due to food preparation, the invention provides tests that test for specific food antigens that are selected on the basis of their stability under processing. Antigens are selected for inclusion in the test not because they are the most clinically relevant allergens, but rather because they are robust to processing (e.g., and do not denature during cooking). Tests of the invention may also test for the most clinically relevant allergens, but importantly, by testing for stable protein products/antigens, the tests report the presence of food residues even after commercial processing.

### TECHNICAL FIELD

The disclosure relates to testing food products for the presence of allergens.

### BACKGROUND

Food allergens are proteins that can cause severe allergic reactions such as anaphylaxis in sensitized individuals. To avoid potentially fatal anaphylactic shock, allergic individuals often seek to strictly avoid even trace amounts of the allergen. Accurate food labeling can help people avoid those ingredients. However, the inadvertent presence of an allergen in food due to cross-contact or labeling error can endanger consumers.

One option to address such situations is to test foods for presence of an allergen. However, some known tests come with inherent limits. For example, some tests use probes that are designed to detect the presence of a known allergen in a food. However, foods are complex products that potentially contain multiple proteins including both allergens and innocuous proteins. Some tests are designed to test for a specific allergen present in food. For example, to address peanut allergy, a test may be provided that tests for Ara h 1, a peanut allergen that is understood to be implicated in the anaphylactic response.

Unfortunately, it may appear at times that such tests do not always detect and report trace amounts of a food allergen in food.

### SUMMARY

The invention provides methods of testing food and food products for the presence of antigens, antigen fragments, or variants that are more stable against food processing or cooking as compared to antigenic variants commonly used for allergen testing. For example, many people exhibit a strong allergic reaction to the Ara h 1 allergen in peanuts. However, the invention is based the recognition that Ara h 1 may be degraded by common preparation processes (e.g., roasting, cooking, etc.), and provides methods of testing for the presence of peanut in food by testing for antigens that are stable through various preparation process. For example, methods of the invention are useful to test for more stable antigens, such as Ara h 2 or Ara h 6, in a food sample to reveal the presence of peanut allergens.

The invention recognizes that food processing, such as cooking, may have different effects on different food proteins. Not only are food proteins differentially affected by processing, some of the most clinically-significant food allergens may be disproportionately broken down by common commercial food preparation methods in a way that makes them undetectable via current methods, yet still potentially potent mediators of allergic reactivity. To avoid a potential false negative allergen detection result on account of food preparation, the invention provides tests that test for specific food antigens that are selected on the basis of their relative stability under processing.

Testing according to the invention may be embodied in scenarios ranging from industrial food processing to simple consumer devices, such as mechanical/electronic devices that fit in a purse or bag and are easy to use table-side, e.g., in a restaurant. Those test devices may be provided with the elements of an immunochemical test, such as the reagents for a lateral flow assay or enzyme-linked immunosorbent assay. The devices optionally may include displays or readouts that show a user a simple binary safe/unsafe result for whether the relevant food is present in a sample.

Within the test device, a test region may include antibodies that are specific to antigens that are selected not because they are the most allergenic, but because they are more stable than other antigens from the same food source. The test region may also include antibodies to other antigens including the most allergenic of the allergens. But importantly, the assay tests for the presence of stable proteins or protein fragments that are indicative of the likelihood of an allergenic response. For example, while a prior table-side test device may test for the presence of Ara h 1 in a food sample, tests of the present invention are focused on more stable antigens or antigen fragments, including, for example, Ara h 2 or a stable fragment, subsequence, or epitope of Ara h 1. In situations where, for example, a baked good is being tested for the inadvertent presence of peanuts, tests according to the invention have a good probability of detecting trace peanut amounts even if baking denatured all of the Ara h 1 in the product.

Thus, the invention provides tests that detect trace amounts of allergen in products, where those tests will be less likely to give false readings due to denaturation of an abundant or dominant antigen. The invention provides food tests in which the antigens being screened for are selected for testing on the basis of stability under a multitude of common processing conditions (even if other, more labile antigens are also included in the test). Tests of the invention are particularly useful in the context of commercial food preparation where peanuts are commonly roasted in a manner that denatures some of the most problematic allergens.

When roasted peanuts are used as an ingredient in a food, the production facility (kitchen, bakery, etc.) may be left with some trace amount of peanut residue, e.g., peanut dust or crumbs. Subsequently-produced food items that are not intended to contain peanuts, such as a salsa, shortbread cookies, or packaged crackers for example, may still include trace amounts of such peanut residue. If those food items are tested for peanut allergens, the test may give a false negative result because the test is specific for an unstable antigen, such as the Ara h 1, that may be denatured in roasting. To address such situations, the invention provides tests specific for the presence of antigens that are selected for inclusion in the test not because those antigens are the most clinically-relevant allergens, but rather because the analyte antigen is robust to processing (e.g., does not denature during roasting or other exposure to heat, pH, enzymes, UV light, etc.) Tests of the invention may also test for the most clinically relevant allergens, but importantly, by testing for stable protein products/antigens, the tests report the presence of food residues even after commercial processing.

In certain aspects, the invention provides a method of testing for an allergen. The method includes introducing a sample from a food product into a test device in which an assay is conducted for an antigen that is substantially resistant to degradation during food processing, and the presence of which is indicative of a potential allergic reaction (variously referred to herein as the “stable antigen”, the “detected antigen” or the “antigen to be detected”). According to the invention, detection of the stable antigen is indicative of the presence of the associated allergen. A second antigen that is unstable during food processing (herein referred to as an “unstable antigen” or “an antigen that is degraded during food processing”, “or a second antigen that is degraded during food processing”) is one that is often denatured by a cooking process. In one example, the antigen to be detected may be from a non-allergenic protein of the food product but its presence is indicative of the presence of the allergen. The detected antigen ideally is thermostable.

In certain embodiments, the detected antigen is Ara h 2 or Ara h 6 and the antigen that is degraded during food processing is Ara h 1 or Ara h 3. The test device may further test for a plurality of other antigens that occur naturally in the food product. For example, for peanuts, the device may test for any number or combination of the Ara h antigens or proteins unique to the peanut species. In some embodiments, the test device tests for a plurality of antigens from a plurality of respective food items (e.g., peanut, milk, egg, and wheat), wherein each antigen is selected because it is relatively more stable than other antigens found in that food source.

The test device may include a plurality of molecular binders attached to a surface that bind to the detected antigen when it is introduced into the test device. For example, the test device may perform an enzyme-linked immunosorbent assay or a lateral flow assay for detection of the antigen. The test device may assay for both the stable antigen and the antigen known to be degraded during food processing (e.g., having the respective antibodies fixed together in a region or spotted onto different regions of the test strip).

In some embodiments, the food product is milk, the stable antigen is from a casein protein, and the antigen that is degraded during food processing is selected from the group consisting of Bos d 4, Bos d 5, Bos d 6, or Bos d 7, Bos d 8, Bos d 9, Bos d 10, Bos d 11, and Bos d 12. In some embodiments, the food product is egg, the stable antigen is from an egg white protein, and the antigen that is degraded during food processing is selected from the group consisting of Gal d 1, Gal d 2, Gal d 3, Gal d 4, Gal d 5, and Gal d 6. In some embodiments, the food product is wheat, the stable antigen is from a glutenin protein, and the antigen known to be degraded during food processing is selected from the group consisting of Tri a 14, Tri a 18, Tri a 19, Tri a 20, Tri a 25, Tri a 26, Tri a 36, tri a 37, Tri a 40, Tri a 41, Tri a 42, Tri a 43 Tri a 44, and Tri a 45.

In certain embodiments, the allergen that is known to be degraded during processing presents a high risk of an anaphylactic response in allergic individuals. The stable antigen is detected more readily in the sample and is a proxy for the potential presence of the allergen that is causative of an allergic response. In immunochemical embodiments, the test device includes antibodies that bind to the antigen. The test device may also include antibodies that bind to the antigen that is typically degraded during food processing. In certain embodiments (i.e., peanut), the stable antigen is one that has been shown to be intact after roasting and the unstable antigen is one that is substantially degraded during roasting.

### DETAILED DESCRIPTION

The invention provides assays for food allergens that that do not substantially degrade during food processing and thus serve as reliable markers of trace allergenic ingredients. The invention employs the insight that during, for example, roasting, allergens of clinical interest may be destroyed. For example, Ara h 1 is substantially denatured when peanuts are roasted. Ara h 1 has been clinically implicated in anaphylaxis with the consequence that some commercially available, even consumer-facing, food tests are designed to test for Ara h 1 as a marker of the presence of peanut residue in a food product. However, those tests may give false negatives because Ara h 1 is often denatured during roasting and many peanuts used in commercial/industrial food production are roasted. Processes that test only for Ara h 1 thus may result in false negatives. To remedy this problem, the invention provides assays that detect antigens that do not get denatured or destroyed in the food processing and preparation process.

Assays of the invention may be embodied in test devices that test for antigens from any food of interest including, for example, milk, egg, fish, crustaceans, shellfish, peanut, tree nuts, wheat, or soy. Methods of the invention may include introducing a sample from a food product into a test device.

The test device may be, for example, a handheld device that includes mechanisms for food processing, antigen detection, result interpretation and data transmission. The device may have a form-factor allowing it to be carried in a purse or bag. Preferably, the device performs all sample processing steps internally, and may be designed to give a quantitative result or, alternatively, a binary positive or negative result.

In some embodiments, the assay inside the device uses the principle of the enzyme-linked immunosorbent assay (ELISA). Monoclonal antibodies may be raised using standard hybridoma techniques. See Council, 1999, Monoclonal Antibody production, National Academic Press, (Washington, D.C.), incorporated by reference. Alternatively, monoclonal antibodies may be discovered from allergic individuals. See Croote, 2018, High-affinity allergen-specific human antibodies cloned from single IgE B cell transcriptomes, Science 362:1306-1309, incorporated by reference. The device may include vials or wells that include capture antibodies and reporter antibodies. Embodiments provide a test device that comes with a vial or reservoir that contains a bead suspension comprising magnetic beads having capture antibodies conjugated to the magnetic beads. A user introduces a food sample into the vial or reservoir. The bead suspension containing the immobilized capture antibodies is then incubated with the extracted food according to provided instructions (e.g., for around 3 min) before re-suspending with horseradish peroxidase (HRP)-conjugated reporter antibodies. The HRP-bead complex can then be mixed with substrate (TMB) and added into a reading device. The device may be provided as a kit with, or with integrated modules for, an extraction device and the extraction buffers and wash solutions in pre-measured volumes. Optionally, a lid of the extraction vial has a magnetic sheathed bar attached to allow for capture of allergen-magnetic beads. This bar allows easy transfer of the antibody-bead complex to the washing/labelling stages and then for loading onto the magnetic electrode, making sample handling easier for the consumer. A reader/sensor includes a microcontroller unit linked with digital-to-analog converters and a controller that controls the potential difference between reference and working electrodes. Alternatively, a reader/sensor includes a microcontroller unit linked with a light source, optional filters, and a detector. Optionally, the reader may be operated via a Bluetooth connection to a smartphone app. The device may include features as described in Lin, 2017, Integrated magneto-chemical sensor for on-site food allergen detection, ACS Nano 11(10):10062-9, incorporated by reference.

In certain embodiments, the assay devices of the invention use the principle of the lateral flow immunoassay (LFIA). The device may have a test strip, to which colloidal gold nanoparticles (red) are conjugated with antibody against the antigen to be tested for. Antibody may be mixed with the nanoparticles at a ratio of 12 μg antibody/ml gold particle (optical density=2) in a basic solution (pH=8.2-8.6) for 30 min at room temperature, and the complex may be dispensed onto the conjugate pad (red pad at the base of strip). A test line containing a second, other antibody may be printed onto a nitrocellulose membrane. The test may include a control line. Components may be assembled into lateral flow strips. E.g., a 25 mm wide nitrocellulose membrane may be glued onto a paper-back card and a wick pad, and the preprinted conjugate pad assembled on top of the rear card with a 2-3 mm overlap. Cover tape may be applied to keep the assembly intact and air was squeezed out by pressing on the assembly. The resulting immunoassay strip is exposed to food sample that has passed through a receiving and processing well of the device. E.g., the device may include a flip cap that opens a to receiving and processing well (of, e.g., about 5 mL volume) into a which a user introduces a food sample. The device may then run homogenizer such as a burr grinder or blade grinder in the well while washing with an extraction solution, e.g., saline or a mild organic solvent such as acetone or ethanol before washing the processed sample to the test strip. Test strips are preferably exposed to the sample, e.g., for about 2 min. Control, hook, and test lines may be included. Intensities may be e captured using a sensor such as a photodiode, CMOS pixel array, or linear-array camera in the device. The device may include features described in U.S. Pub. 2016/0266083 A1, incorporated by reference.

A simple processor (e.g., a field programmable gate array or application specific integrated circuit) may read the sensor and report the presence and/or quantity of the analyte(s), e.g., give an output showing that the antigen was present in the input sample.

In some embodiments of the assay device, the device includes a reusable testing instrument and a disposable testing unit. Users put a food sample into the testing unit, e.g., into the receiving well, and fasten (e.g., screw down) a lid. Once the lid is fastened, the testing unit mechanically grinds the food sample and releases an extraction solution. E.g., the user may place the testing unit into the testing instrument and press a button to have the instrument initiate mixing in the unit. Optionally, fully attaching the unit to the instrument causes the opening of an internal valve or passage allowing liquid from the extraction chamber to flow onto the LFIA strip. Mechanical features may be as described in U.S. Pat. No. 9,939,431 B2, incorporated by reference. The assay device assays the food sample for at least one antigen that occurs naturally in the food product and is more stable during food processing as compared to another antigen that may be present in the food product. Using, e.g., the sensor and the processor, the device reports a risk of the allergen being in the food product when the stable (detected) antigen is detected. In certain embodiments, the unstable allergen is one that is often denatured by a cooking process and the stable antigen is one that is rarely denatured by the cooking process. E.g., the detected antigen may be a thermo-stable antigen of a non-allergenic protein of the food product. In some embodiments, the antigen that is tested for is Ara h 2 and/or Ara h 6 and the unstable antigen is Ara h 1 and or Ara h 3 (which may also be tested for).

The test device may test for a plurality of antigens that occur naturally in the food product. Table 1 lists antigens that may be tested for.

TABLE 1
antigens that may be tested for; reproduced from Sharma, 2017, Detection of allergen
markers in food: analytical methods, FDA Papers 6:65-121, incorporated by reference.

| | | | |
|---|---|---|---|
| Allergen | Biochemical name | Allergen | Biochemical name |

MILK

| | | | |
|---|---|---|---|
| Bos d 4 | α-lactalbumin | Bos d 9 | α-S1-casein |
| Bos d 5 | β-lactoglobulin | Bos d 10 | α-S2-casein |
| Bos d 6 | Serum albumin | Bos d 11 | β-casein |
| Bos d 7 | Immunoglobulin | Bos d 12 | K-casein |
| Bos d 8 | Casein | | |

EGG

| | | | |
|---|---|---|---|
| Allergen | Biochemical name | Allergen | Biochemical name |
| Gal d 1 | Ovomucoid | Gald 4 | Lysozyme C |
| Gal d 2 | Ovalbumin | Gald 5 | Serum albumin |
| Gal d 3 | Ovotransferrin | Gald 6 | YGP4 |

FISH

| Allergen | Biochemical name | Allergen | Biochemical name |
|---|---|---|---|
| Yellowfin tuna | | Atlantic cod | |
| Thu a 1 | β-parvalbumin | Gad m 1 | β-parvalbumin |
| Thu a 2 | β-enolase | Gad m 2 | β-enolase |
| Thu a 3 | Aldolase A | Gad m 3 | Aldolase A |
| Atlantic salmon | | Baltic cod | |
| Sal s 1 | β-parvalbumin 1 | Gad c 1 | β-parvalbumin |
| Sal s2 | β-Enolase | | |
| Sal s 3 | Aldolase A | | |

CRUSACEAN SHELLFIS

| Allergen | Biochemical name | Allergen | Biochemical name |
|---|---|---|---|
| Black tiger shrimp | | American lobster | |
| Pen m l | Tropomyosin | Hom a 1 | Tropomyosin |
| Pen m 2 | Arginine kinase | Hom a 3 | Myosin light chain 2 |
| Pen m 3 | Myosin light chain 2 | Hom a 6 | Troponin C |
| Pen m 4 | Sarcoplasmic Ca | Spiny lobster | |
| | binding protein | | |
| Pen m 6 | Troponin C | Pan s 1 | Tropomyosin |
| Crab | | | |
| Cha f 1 | Tropomyosin | | |

PEANUT

| | | | |
|---|---|---|---|
| Allergen | Biochemical name | Allergen | Biochemical name |
| Ara h 1 | 7S globulin | Ara h 10 | 16 kDa oleosin |
| Ara h 2 | 2S albumin | Ara h 11 | 14 kDa oleosin |
| Ara h 3 | 11S globulin | Ara h 12 | Defensin |
| Ara h 4 | renamed Ara h 3.02 | Ara h 13 | Defensin |
| Ara h 5 | Profilin | Ara h 14 | Oleosin |
| Ara h 6 | 2S albumin | Ara h 15 | Oleosin |
| Ara h 7 | 2S albumin | Ara h 16 | nsLTP |
| Ara h 8 | PR-10 | Ara h 17 | nsLTP |
| Ara h 9 | nsLTP | | |

TREE NUTS

| Allergen | Biochemical name | Allergen | Biochemical name |
|---|---|---|---|
| Almond | | Brazil nut | |
| Pru du 3 | nsLTP 1 | Ber e 1 | 2S albumin |
| Pru du 4 | Profilin | Ber e 2 | 11S globulin |
| Pru du 5 | 60s acidic ribosomal | Hazelnut | |
| | protein P2 | | |
| Pru du 6 | Amandin, US | Cor a 1 | PR-10 |
| | globulin | | |
| Cashew nut | | Cor a 2 | Profilin |
| Ana o 1 | 7S globulin | Cor a 8 | nsLTP 1 |
| Ana o 2 | 11S globulin | Cor a 9 | 11S globulin |
| Ana o 3 | 2S albumin | Cor all | 7S globulin |
| Pecan | | Cor a 12 | 17 kDa oelosin |
| Cari 1 | 2S albumin | Cor a 13 | 14-16 kDa oleosin |
| Car i 2 | 7S globulin | Cor a 14 | 2S albumin |
| Car i 4 | 11S globulin | English walnut | |
| Pistachio | | Jug r 1 | 2S albumin |
| Pis v 1 | 2S albumin | Jug r 2 | 7S globulin |
| Pis v 2 | 11S globulin | Jug r 3 | nsLTP 1 |
| Pis v 3 | 7S globulin | Jug r 4 | 11S globulin |
| Pis v 4 | Manganese | Jug r 5 | PR-10 |
| | superoxide dismutase | | |
| Pis v 5 | 11S globulin | | |

WHEAT

| | | | |
|---|---|---|---|
| Allergen | Biochemical name | Allergen | Biochemical name |
| Tri a 14 | nsLTPl | Tri a 3 | purothionin |
| Tri a 18 | Agglutinin isolectin 1 | Tri a 40 | amylase inhibitor |
| Tri a 1 | Omega-5 gliadin | Tri a 41 | Mitochondrial |
| | | | ubiquitin ligase |
| | | | activator of NFKB 1 |
| Tri a 2 | Gliadin | Tri a 42 | Hypothetical protein |
| Tri a 25 | Thioredoxin | Tri a 43 | Hypothetical protein |
| Tri a 26 | High molecular | Tri a 44 | Endosperm transfer |
| | weight glutenin | | cell specific PR60 |
| | | | precursor |
| Tri a 36 | Low molecular | Tri a 45 | Elongation factor 1 |
| | weight glutenin | | (EIF1) |
| | GluB3-23 | | |

SOY

| | | | |
|---|---|---|---|
| Allergen | Biochemical name | Allergen | Biochemical name |
| Gly m 3 | Profilin | Gly m 6 | 11S globulin |
| Gly m 4 | PR-10 | Gly m 7 | Seed biotinylated |
| | | | protein |
| Gly m 5 | 7S globulin | Gly m 8 | 2S albumin |

In various embodiments, the test device tests for a plurality of antigens from a plurality of respective food items, wherein each antigen is selected because it is relatively more stable than other allergens found in that food source. Optionally, the test device includes a plurality of molecular binders (e.g., antibodies) attached to a surface that bind to the stable antigen when it is introduced into the test device. For example, the test device may be useful for performing an enzyme-linked immunosorbent assays or a lateral flow assay for the stable antigen.

The test device may include antibodies that bind specifically to the stable antigen and capture it for detection. For example, antibodies may be raised against, or characterized to be specific to, antigens indicative of any of the allergens shown in Table 1 and included in the test device. The antigen that is tested for is selected because it is more robust to processing (e.g., thermostable, and less likely to denature) than a known, clinically-important allergen (e.g., one that is thermolabile). In some cases, the unstable allergen is known to present a high risk of an anaphylactic response in allergic individuals.

The invention provides methods of testing food for antigens that are more stable to food processing than antigens that, while diagnostically-relevant, are often degraded during processing. When clinically-significant allergens are disproportionately broken down by common preparation methods, the presence of certain food ingredients may be “masked” to some tests. To prevent clinically problematic allergens from being masked by food preparation, the invention provides tests that test for specific food antigens that are selected on the basis of their stability under processing. Antigens are selected for inclusion in the test not because they are the most clinically relevant allergens, but rather because they are robust to processing (e.g., and do not denature during cooking). Tests of the invention may also test for the most clinically relevant allergens, but importantly, by testing for stable protein products/antigens, the tests report the presence of food residues even after commercial processing.

In fact, embodiments of the invention may address a specific phenomenon by which global allergy incidence is unevenly distributed in a manner that may be linked to predominant geographical cooking practices. It may be found that peanuts are processed in North America and other parts of the Western world primarily by roasting, while peanuts in Asia and other parts of the Eastern world are primarily processed by boiling or frying. It may be found that the times and temperatures of those different cooking practices have different effects on allergen stability with a consequence of early childhood immune sensitization having different patterns across the globe. It may be found that Ara h 1 or Ara h 3 are denatured by roasting in such a way that some populations are not desensitized to it and develop proportionally higher incidence of allergy. However, some commercial tests may employ antibodies specific to Ara h 1 or Ara h 3 with the consequence that the roasting masks the presence of trace peanut amounts to the test due to denaturation. Tests of the invention include assays that test for antigens other than Ara h 1 or Ara h 3 to detect trace amounts of peanut without requiring the specific molecular detection of Ara h 1 or Ara h 3.

It is important to note that assays of the invention may find particular utility in, and are intended to be used for, detecting trace amounts of ingredients in food products for which the ingredient is not intended to be an ingredient. For example, tests of the invention are useful to detect trace amounts of peanut dust in foods that are not expected to include any peanuts. Tests of the invention may be used, for example, to test for trace amounts of peanut in foods and environments such as in cotton candy at a fair, or in butter cookies at a bakery, or in a gelatin desert at a social gathering, or in a fruit smoothie from a juice bar.

What is claimed is:

1. A method of testing for an allergen, the method comprising:
introducing a sample from a food product into a test device;
conducting an assay in the test device to detect an antigen that is substantially resistant to degradation during food processing, and the presence of which is indicative of a potential allergic reaction; and
reporting the presence of an allergen in the food product as a result of detection of the antigen.

2. The method of claim 1, wherein the antigen is an isotype of a second antigen that is degraded during food processing.

3. The method of claim 1, wherein the antigen is Ara h 2, Ara h 6 or a combination of Ara h 2 and Ara h6.

4. The method of claim 2, wherein the antigen that is degraded is Ara h 1,Ara h 3, or a combination of Ara h 1 and Ara h 3.

5. The method of claim 1, wherein the test device further tests for a plurality of antigens that occur naturally in the food product.

6. The method of claim 1, wherein the test device tests for a plurality of antigens from a food item, wherein each antigen is selected because it is relatively more stable than other allergens found in said food item.

7. The method of claim 1, wherein the test device includes a plurality of molecular binders attached to a surface that bind to the antigen when the antigen is introduced into the test device.

8. The method of claim 1, wherein the test device is adapted to perform an enzyme-linked immunosorbent assay or a lateral flow assay for the antigen.

9. The method of claim 1, wherein the test device detects the antigen and the second antigen that is degraded during food processing.

10. The method of claim 2, wherein the food product is milk, the antigen is from a casein protein, and the second antigen that is degraded during food processing is selected from the group consisting of Bos d 4, Bos d 5, Bos d 6, Bos D 7, Bos D 8, Bos D 9, Bos d 10, Bos d 11, and Bos d 12.

11. The method of claim 2, wherein the food product is egg, the antigen is from an egg white protein, and the second antigen that is degraded during food processing is selected from the group consisting of Gal d 1, Gal d 2, Gal d 3, Gal d 4, Gal d 5, and Gal d 6.

12. The method of claim 2, wherein the food product is wheat, the antigen is from a glutenin protein, and the second antigen that is degraded during food processing is selected from the group consisting of Tri a 14, Tri a 18, Tri a 19, Tri a 20, Tri a 25, Tri a 26, Tri a 36, tri a 37, Tri a 40, Tri a 41, Tri a 42, Tri a 43 Tri a 44, and Tri a 45.

13. The method of claim 1, wherein the second antigen that is degraded during food processing is known to present a high risk of an anaphylactic response in allergic individuals.

14. The method of claim 1, wherein the antigen is suspected to present a lower risk of anaphylactic response in allergic individuals and has greater thermal stability than the second antigen that is degraded during food processing.

15. The method of claim 1, wherein the test device comprises antibodies that bind to the antigen.
